(19) United States
(12) Patent Application Publication
(10) Pub. No.: US 2019/0259500 A1
(43) Pub. Date: Aug. 22, 2019

(54) Health Behavior Change for Intelligent Personal Assistants

(71) Applicant: International Business Machines Corporation, Armonk, NY (US)

(72) Inventors: Alaa Abou Mahmoud, Dracut, MA (US); Paul R. Bastide, Boxford, MA (US); Fang Lu, Billerica, MA (US)

(21) Appl. No.: 15/899,736

(22) Filed: Feb. 20, 2018

(51) Int. Cl. G16H 50/30; G06F 17/30; G10L 15/22; H04L 29/08

(52) U.S. Cl. CPC G16H 50/30; H04L 67/22; G10L 15/22; G06F 17/30976

(57) ABSTRACT

A mechanism is provided in a data processing system comprising a processor and a memory, the memory comprising instructions that are executed by the processor to specifically configure the processor to implement an intelligent personal assistant with a health behavior change engine. A user state monitoring component monitors a user state received from an intelligent personal assistant. The user state monitoring component generates a user data model data structure that represents a pattern of behavior of the user based on the monitored user state. A user state change detection component detects a change in user state. A state change analysis component analyzes the change in user state to determine whether to activate a dialog. A dialog generation component generates a health-based conversation dialog in response to the state change analysis component determining to activate the dialog. A communication component executes with the user via the intelligent personal assistant using the health-based conversation dialog.

### BACKGROUND

The present application relates generally to an improved data processing apparatus and method and more specifically to mechanisms for health behavior change for intelligent personal assistants.

A virtual assistant or intelligent personal assistant is a software agent that can perform tasks or services for an individual. Sometimes the term “chatbot” is used to refer to virtual assistants generally or specifically those accessed by online chat. The capabilities and usage of virtual assistants is expanding rapidly, with new products entering the market. Intelligent personal assistants may be integrated into many types of platforms. For example, intelligent personal assistants may be integrated into devices like smart speakers, instant messaging apps, mobile operating systems (OS), instant messaging platforms, smart watches, appliances, cars, Web sites, interactive voice response (IVR) systems, etc.

Intelligent personal assistants can provide a wide variety of services, which grow by the day. These include: providing information such as weather and news, setting an alarm, making to-do lists and shopping lists, playing music from streaming services, playing radio stations, reading audio books, playing shows or movies on televisions, streaming video from streaming services, buying items from online retailers, complementing and/or replacing human customer service, etc.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided in a data processing system comprising a processor and a memory, the memory comprising instructions that are executed by the processor to specifically configure the processor to implement an intelligent personal assistant with a health behavior change engine. The method comprises monitoring, by a user state monitoring component executing within the health behavior change engine, a user state received from an intelligent personal assistant. The method further comprises generating, by the user state monitoring component, a user data model data structure that represents a pattern of behavior of the user based on the monitored user state. The method further comprises detecting, by a user state change detection component executing within the health behavior change engine, a change in user state. The method further comprises analyzing, by a state change analysis component executing within the health behavior change engine, the change in user state to determine whether to activate a dialog. The method further comprises generating, by a dialog generation component executing within the health behavior change engine, a health-based conversation dialog in response to the state change analysis component determining to activate the dialog. The method further comprises communicating, by a communication component executing within the health behavior change engine, with the user via the intelligent personal assistant using the health-based conversation dialog.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example diagram of a distributed data processing system in which aspects of the illustrative embodiments may be implemented;

FIG. 2 is an example block diagram of a computing device in which aspects of the illustrative embodiments may be implemented;

FIG. 3 depicts a pictorial representation of an example data processing system with an intelligent personal assistant in which aspects of the illustrative embodiments may be implemented;

FIG. 4 is a block diagram of a health behavior change engine in accordance with an illustrative embodiment; and

FIG. 5 is a flowchart illustrating operation of a health behavior change engine in accordance with an illustrative embodiment.

### DETAILED DESCRIPTION

Untreated acute health issues contribute to the rise in health care costs and absenteeism related productivity losses. As shown in the research, early education, intervention, and action produce better outcomes. Busy work and a fast lifestyle lead people to minimize the attention to health issues, which often result in more severe consequences. For example, a busy executive may have a packed meeting schedule. The executive may come down with a cold and fail to reschedule her meetings. If the executive's cold goes untreated, more severe outcomes could result, such as sinus infection. A change in health behaviors could prevent worsening heal conditions and loss of productivity.

The illustrative embodiments provide mechanisms within intelligent personal assistants that initiate and support health behavior changes by detecting a need to activate based on changes to user state, analyzing the changed state to predict a future routine change, preparing a dialog based on the routine change, and communicating via the intelligent personal assistant at the predicted future routine change with the given dialog. The changes may be detected based on behavior changes, driving pattern changes, searches (health-related), etc. The communication may be scoped to a specific period (e.g., for a cold, seven days). The mechanisms of the illustrative embodiments may activate only when multiple routine items are not met or the user fails to interact with calendar meetings.

Consider an example scenario of user state determination. John is a smart phone user and has configured an intelligent personal assistant to work with his voice signature. John gets ready for work and asks the intelligent personal assistant, “How long does it take to get to Littleton, Massachusetts? What is the weather there?” The mechanism of the illustrative embodiment captures John's average state and common questions. For example, John often interacts with the intelligent personal assistant while preparing for work, commuting to work, driving home, and querying about traffic conditions on Monday through Friday. John frequently asks, “Where would be a good place to attend happy hour?” on Fridays. John asks, “What new movies are in the theater?” on Saturday afternoon and Sunday afternoon. The mechanism of the illustrative embodiment captures this relevant information about the user and saves the analyzed pattern into a user data model data structure as the user's state pattern.

Now consider an example scenario of a user state change interaction. The mechanism of the illustrative embodiment identifies that John has not asked anything about happy hour on a given Friday afternoon and identifies that John did not ask anything about whether there is a good new movie coming out on Saturday afternoon. The mechanism identifies the changed user state (behavior that deviates from pattern) from the user. The mechanism prepares questions that replay previous questions to John: “Would you like to know about today's weather?” “Would you like to know how long it takes to reach Littleton?” The mechanism checks the calendar for John and determines he is not traveling. The mechanism queries a flu/cold map. The mechanism then prepares the following dialog: “Good morning, John, there is a small percentage of flu/cold in your area. Would you like me to route you to a pharmacy?” The mechanism then prepares questions to check with John on his physiological and emotional state as follows: “How do you feel today” or “Are you alright?” or “Are you tired?”

Then, John picks up his smart phone, and the accelerometer indicates the smart phone is in use. The mechanism prompts John with the prepared questions and monitors the dialog. John responds, and the mechanism may make further inquiries and resolve down to a specific sickness or disease. For example, “Would you like directions to a pharmacy to get cold medicine?” or “Do you need to make a doctor appointment?”

Before beginning the discussion of the various aspects of the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

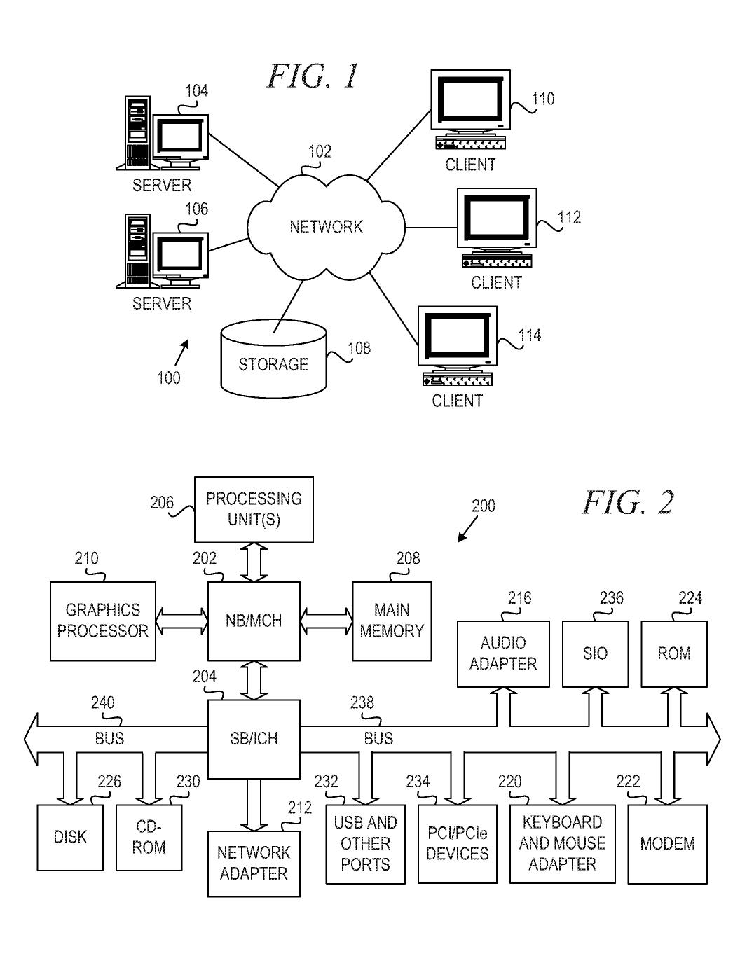

The illustrative embodiments may be utilized in many different types of data processing environments. In order to provide a context for the description of the specific elements and functionality of the illustrative embodiments, FIGS. 1 and 2 are provided hereafter as example environments in which aspects of the illustrative embodiments may be implemented. It should be appreciated that FIGS. 1 and 2 are only examples and are not intended to assert or imply any limitation with regard to the environments in which aspects or embodiments of the present invention may be implemented. Many modifications to the depicted environments may be made without departing from the spirit and scope of the present invention.

FIG. 1 depicts a pictorial representation of an example distributed data processing system in which aspects of the illustrative embodiments may be implemented. Distributed data processing system 100 may include a network of computers in which aspects of the illustrative embodiments may be implemented. The distributed data processing system 100 contains at least one network 102, which is the medium used to provide communication links between various devices and computers connected together within distributed data processing system 100. The network 102 may include connections, such as wire, wireless communication links, or fiber optic cables.

In the depicted example, server 104 and server 106 are connected to network 102 along with storage unit 108. In addition, clients 110, 112, and 114 are also connected to network 102. These clients 110, 112, and 114 may be, for example, personal computers, network computers, or the like. In the depicted example, server 104 provides data, such as boot files, operating system images, and applications to the clients 110, 112, and 114. Clients 110, 112, and 114 are clients to server 104 in the depicted example. Distributed data processing system 100 may include additional servers, clients, and other devices not shown.

In the depicted example, distributed data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, the distributed data processing system 100 may also be implemented to include a number of different types of networks, such as for example, an intranet, a local area network (LAN), a wide area network (WAN), or the like. As stated above, FIG. 1 is intended as an example, not as an architectural limitation for different embodiments of the present invention, and therefore, the particular elements shown in FIG. 1 should not be considered limiting with regard to the environments in which the illustrative embodiments of the present invention may be implemented.

As shown in FIG. 1, one or more of the computing devices, e.g., server 104, may be specifically configured to implement a health behavior change engine in an intelligent personal assistant. The configuring of the computing device may comprise the providing of application specific hardware, firmware, or the like to facilitate the performance of the operations and generation of the outputs described herein with regard to the illustrative embodiments. The configuring of the computing device may also, or alternatively, comprise the providing of software applications stored in one or more storage devices and loaded into memory of a computing device, such as server 104, for causing one or more hardware processors of the computing device to execute the software applications that configure the processors to perform the operations and generate the outputs described herein with regard to the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general purpose computing device. Moreover, as described hereafter, the implementation of the mechanisms of the illustrative embodiments improves the functionality of the computing device and provides a useful and concrete result that facilitates health behavior change for intelligent personal assistants.

As noted above, the mechanisms of the illustrative embodiments utilize specifically configured computing devices, or data processing systems, to perform the operations for a health behavior change engine in an intelligent personal assistant. These computing devices, or data processing systems, may comprise various hardware elements which are specifically configured, either through hardware configuration, software configuration, or a combination of hardware and software configuration, to implement one or more of the systems/subsystems described herein. FIG. 2 is a block diagram of just one example data processing system in which aspects of the illustrative embodiments may be implemented. Data processing system 200 is an example of a computer, such as server 104 in FIG. 1, in which computer usable code or instructions implementing the processes and aspects of the illustrative embodiments of the present invention may be located and/or executed so as to achieve the operation, output, and external effects of the illustrative embodiments as described herein.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 may be connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 may be connected to SB/ICH 204.

An operating system runs on processing unit 206. The operating system coordinates and provides control of various components within the data processing system 200 in FIG. 2. As a client, the operating system may be a commercially available operating system such as Microsoft® Windows 7®. An object-oriented programming system, such as the Java™ programming system, may run in conjunction with the operating system and provides calls to the operating system from Java™ programs or applications executing on data processing system 200.

As a server, data processing system 200 may be, for example, an IBM eServer™ System p® computer system, Power™ processor based computer system, or the like, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and may be loaded into main memory 208 for execution by processing unit 206. The processes for illustrative embodiments of the present invention may be performed by processing unit 206 using computer usable program code, which may be located in a memory such as, for example, main memory 208, ROM 224, or in one or more peripheral devices 226 and 230, for example.

A bus system, such as bus 238 or bus 240 as shown in FIG. 2, may be comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, may include one or more devices used to transmit and receive data. A memory may be, for example, main memory 208, ROM 224, or a cache such as found in NB/MCH 202 in FIG. 2.

As mentioned above, in some illustrative embodiments the mechanisms of the illustrative embodiments may be implemented as application specific hardware, firmware, or the like, application software stored in a storage device, such as HDD 226 and loaded into memory, such as main memory 208, for executed by one or more hardware processors, such as processing unit 206, or the like. As such, the computing device shown in FIG. 2 becomes specifically configured to implement the mechanisms of the illustrative embodiments and specifically configured to perform the operations and generate the outputs described hereafter with regard to the health behavior change engine.

Those of ordinary skill in the art will appreciate that the hardware in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

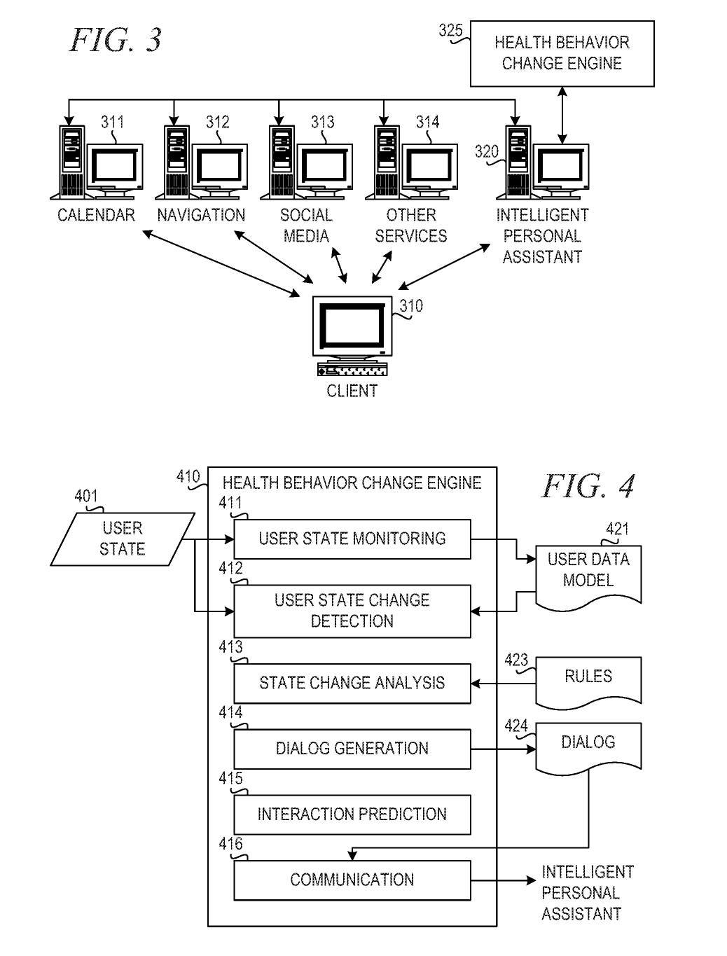

FIG. 3 depicts a pictorial representation of an example data processing system with an intelligent personal assistant in which aspects of the illustrative embodiments may be implemented. Client 310 connects to a plurality of apps or services 311-314, including calendar 311, navigation 312, social media 313, and other services 314. Calendar 311 and navigation 312 may be local apps or cloud-based services, for example. Social media 313 may include one or more online services. Other services 314 may include other local apps or other online or cloud-based services, such as electronic mail, music streaming, video streaming, image storage, text or video chat, note taking, games, news services, travel services, shopping sites, food ordering services, or the like.

Client 310 may also connect to intelligent personal assistant 320, which may be a local app or cloud-based service. In accordance with one embodiment, a user at client 310 may give permission to intelligent personal assistant to access one or more of the plurality of apps or services 311-314. The user at client 310 may access apps or services 311-314 through intelligent personal assistant 320, such as by voice or text input. For instance, the user may activate intelligent personal assistant using a voice command to a smart speaker or smart phone. The user may tell intelligent personal assistant 320 to make a new appointment, find directions to a location, read news, play music, order household items or groceries, etc.

Intelligent personal assistant 320 may access the user content in the apps or services 311-314. For instance, intelligent personal assistant 320 may access calendar 311 to see if there are any upcoming appointments. The user content may be stored local on client 310 or in the cloud. In addition, intelligent personal assistant 320 may monitor apps or services 311-314 for new user states or behaviors. For example, intelligent personal assistant 320 may detect a new appointment being added to calendar app or service 311 or a new destination or route being entered into navigation app or service 312. Intelligent personal assistant 320 may provide capabilities based on past and current states or behaviors and future events learned from apps or services 311-314. For instance, in response to the user at client device 310 entering a new appointment with a location in a different city, intelligent personal assistant may offer to present current price information for flights to that city.

In accordance with the illustrative embodiment, intelligent personal assistant includes health behavior change engine 325, which may be embodied within intelligent personal assistant or may be separate application or plug-in, often referred to as a “skill.” Health behavior change engine 325 detects a need to activate based on changes to user state. Health behavior change engine 325 monitors the user's communication pattern across time and develops a user data model. For example, health behavior change engine 325 may detect appointments, driving patterns, etc. Health behavior change engine 325 uses frequent pattern mining to identify a pattern to develop the user data model. Health behavior change engine 325 may seed the patterns based on patterns observed by people in a local geography or business or by similar users matching a specific profile.

Health behavior change engine 325 monitors for changes in communication pattern or behavior. For example, health behavior change engine 325 may detect changes in interaction patterns, changed appointments, new driving patterns, missed appointments, health-related searches, health-related social media posts, etc. Health behavior change engine 325 may determine whether a predetermined number of routine items are not met or communicated with, such as missed meetings, emails without a reply, etc. In one embodiment, health behavior change engine 325 may be enabled/disabled during particular periods of time or in particular locations (e.g., home or work). For instance, health behavior change engine 325 may detect whether the user is on vacation.

Health behavior change engine 325 analyzes the changed state or behavior to predict a future routine change. Health behavior change engine 325 monitors interactions with the intelligent personal assistant 320. Health behavior change engine 325 may determine that an interaction does not occur at a specified time within a threshold (e.g., 15 minutes, 30 minutes, etc.). Health behavior change engine 325 may retrieve the next routine item in order. Health behavior change engine 325 may interact only with future routine changes that are not met, as it is an ideal time for a dialog with the user.

Health behavior change engine 325 prepares a dialog based on a detected routine change. Health behavior change engine 325 retrieves a next predicted changed item and converts the routine item into a question or prompts the user with details related to the question. Health behavior change engine 325 may fill out a template with a question. For example, health behavior change engine 325 may use slot filler templates to form questions or statements for the dialog with information filled in based on the user state change, behavior change, or predicted routine change. In one embodiment, health behavior change engine 325 searches a central repository to match whether there is any potential health related concerns that should be raised. If health behavior change engine 325 determines there is a health related concern, then health behavior change engine 325 prepares a dialog and automatically engages the user for inquiry and to make necessary suggestions.

Health behavior change engine 325 communicates via intelligent personal assistant 320 at the predicted future routine change with the given dialog. Health behavior change engine 325 composes an intelligent personal assistant prompt to prompt the user and continue the dialog with the missed actions or possible actions. The communication may be scoped to a specific time period. For instance, if the related health concern is an increased incidence of the cold virus, then the communication may be scoped to seven days.

Health behavior change engine 325 may check the local area to determine if other users are in the area. For example, to protect privacy, health behavior change engine 325 may monitor audio input or detect location to determine whether other users may be nearby. Thus, if the user is in a crowded place, then health behavior change engine 325 may delay communication with the user until a later time when the user is likely to be alone.

FIG. 4 is a block diagram of a health behavior change engine in accordance with an illustrative embodiment. Health behavior change engine 410 receives user state information 401 from apps or services via an intelligent personal assistant. Health behavior change engine includes user state monitoring component 411, user state change detection component 412, state change analysis component 413, dialog generation component 414, interaction prediction component 415, and communication component 416. User state monitoring component 411 monitors user state 401 and detects interaction and communication patterns. For example, user state monitoring component 411 may detect patterns in calendar appointments, driving routes, social media interactions, searches, media consumption, etc. User state monitoring component 411 generates user data model data structure 421, which represents expected patterns of user behavior.

User state change detection component 412 monitors user state 401 and detects when user state 401 deviates from expected patterns in user data model 421. For example, if the user regularly asks the intelligent personal assistant for a good place for happy hour on Fridays, then this information will be represented in user data model 421. User state change detection component 412 would then detect when a Friday passes without the user asking the intelligent personal assistant about happy hour.

State change analysis component 413 detects a need to activate based on changes to the user's state 401. State change analysis component 413 may apply rules 423 to changes in the user's state or behavior. For example, rules 423 may cause state change analysis component 413 to activate in response to an important meeting being missed, in response to a predetermined number of appointments being changed, in response to driving navigation being changed in destination, route, or time, etc. For example, if the user travels home from work in the middle of the work day, this may be indicative of an illness. The rules may be configurable by the user.

State change analysis component 413 may also determine whether there is a health concern. For example, state change analysis component 413 may search a central repository to determine whether there is an increased incidence of virus in the user's geographic location. State change analysis component 413 may also look at the user's emails, social media posts, or searches for indicators of a medical condition.

Dialog generation component 414 prepares dialog 424 for communicating with the user. Dialog generation component 414 may use templates to generate questions or statements with information about a detected health concern. Dialog 424 may ask the user questions to help identify a medical condition or may inform the user of potential health concerns based on the detected user state change. For example, dialog 424 may ask the user if he or she is tired or achy. Dialog 424 may ask if the user wishes to make an appointment with a doctor or stop at a pharmacy. Dialog 424 may also make suggestions, such as delaying appointments, getting rest, or the like.

Interaction prediction component 415 analyzes the changed state to predict a future routine item. In one embodiment, interaction prediction component 414 may examine user data model 421 to determine a next routine item in order. Interaction prediction component 415 determines a next interaction with the intelligent personal assistant at which time health behavior change engine 410 will communicate with the user using the dialog 424.

Communication component 416 communicates with the user via the intelligent personal assistant. Communication component 416 prompts the user and conducts a dialog with the user based on dialog 424. Communication component 416 may scope the communication to a specific time period or to a specific location.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

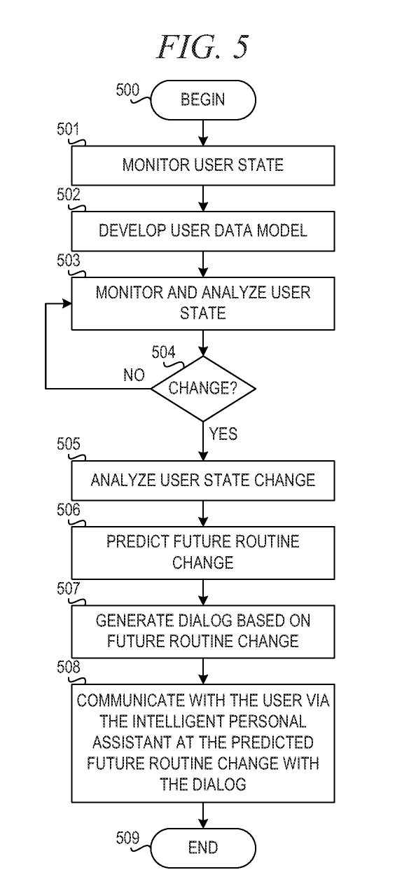

FIG. 5 is a flowchart illustrating operation of a health behavior change engine in accordance with an illustrative embodiment. Operation begins (block 500), and the health behavior change engine monitors the user state (block 501) and develops a user data model (block 502). The health behavior change engine then monitors and analyzes the user state (block 503) and determines whether there is a change in user state (block 504). If there is not a change in user state, then operation returns to block 503 to monitor and analyze user state until there is a change in user state.

If there is a change in user state in block 504, then the health behavior change engine analyzes the user state change (block 505). The health behavior change engine predicts a future routine change (block 506). The health behavior change engine generates a dialog based on the future routine change (block 507). Then, the health behavior change engine communicates with the user via the intelligent personal assistant at the predicted future routine change with the dialog (block 508). Thereafter, operation ends (block 509).

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions that are executed by the processor to specifically configure the processor to implement an intelligent personal assistant with a health behavior change engine, the method comprising:
monitoring, by a user state monitoring component executing within the health behavior change engine, a user state received from an intelligent personal assistant;
generating, by the user state monitoring component, a user data model data structure that represents a pattern of behavior of the user based on the monitored user state;
detecting, by a user state change detection component executing within the health behavior change engine, a change in user state;
analyzing, by a state change analysis component executing within the health behavior change engine, the change in user state to determine whether to activate a dialog;
generating, by a dialog generation component executing within the health behavior change engine, a health-based conversation dialog in response to the state change analysis component determining to activate the dialog; and
communicating, by a communication component executing within the health behavior change engine, with the user via the intelligent personal assistant using the health-based conversation dialog.

2. The method of claim 1, wherein the user state is received from an app or service within a plurality of apps or services.

3. The method of claim 2, wherein the plurality of apps or services comprise at least one local application.

4. The method of claim 2, wherein the plurality of apps or services comprise at least one cloud-based service.

5. The method of claim 1, wherein generating the user data model data structure comprises detecting patterns in calendar appointments, driving routes, social media interactions, searches, or media consumption.

6. The method of claim 1, wherein the change in user state comprises a change in interaction pattern, a changed appointment, a new driving pattern, a missed appointment, a health-related search, or a health-related social media post.

7. The method of claim 1, wherein analyzing the change in user state to determine whether to activate a dialog comprises applying a set of rules to the change in user state.

8. The method of claim 1, wherein generating, by a dialog generation component executing within the health behavior change engine, a health-based conversation dialog comprises using slot filler templates to form questions or statements for the dialog with information filled in based on the user state change, a behavior change, or a predicted routine change.

9. The method of claim 1, further comprising predicting a future routine change and communicating with the user at the predicted future routine change.

10. The method of claim 1, wherein communication with the user may be scoped to a specific time period or a specific location.

11. A computer program product comprising a computer readable storage medium having a computer readable program stored therein, wherein the computer readable program, when executed on at least one processor of a data processing system, causes the data processing system to implement an intelligent personal assistant with a health behavior change engine, wherein the computer readable program causes the data processing system to:
monitor, by a user state monitoring component executing within the health behavior change engine, a user state received from an intelligent personal assistant;
generate, by the user state monitoring component, a user data model data structure that represents a pattern of behavior of the user based on the monitored user state;
detect, by a user state change detection component executing within the health behavior change engine, a change in user state;
analyze, by a state change analysis component executing within the health behavior change engine, the change in user state to determine whether to activate a dialog;
generate, by a dialog generation component executing within the health behavior change engine, a health-based conversation dialog in response to the state change analysis component determining to activate the dialog; and
communicate, by a communication component executing within the health behavior change engine, with the user via the intelligent personal assistant using the health-based conversation dialog.

12. The computer program product of claim 11, wherein the user state is received from an app or service within a plurality of apps or services.

13. The computer program product of claim 12, wherein the plurality of apps or services comprise at least one local application.

14. The computer program product of claim 12, wherein the plurality of apps or services comprise at least one cloud-based service.

15. The computer program product of claim 11, wherein generating the user data model data structure comprises detecting patterns in calendar appointments, driving routes, social media interactions, searches, or media consumption.

16. The computer program product of claim 11, wherein the change in user state comprises a change in interaction pattern, a changed appointment, a new driving pattern, a missed appointment, a health-related search, or a health-related social media post.

17. The computer program product of claim 11, wherein analyzing the change in user state to determine whether to activate a dialog comprises applying a set of rules to the change in user state.

18. The computer program product of claim 11, wherein generating, by a dialog generation component executing within the health behavior change engine, a health-based conversation dialog comprises using slot filler templates to form questions or statements for the dialog with information filled in based on the user state change, a behavior change, or a predicted routine change.

19. The computer program product of claim 11, wherein communication with the user may be scoped to a specific time period or a specific location.

20. An apparatus comprising:
a processor; and
a memory coupled to the processor, wherein the memory comprises instructions which, when executed by the processor, cause the processor to implement an intelligent personal assistant with a health behavior change engine, wherein the instructions cause the processor to:
monitor, by a user state monitoring component executing within the health behavior change engine, a user state received from an intelligent personal assistant;
generate, by the user state monitoring component, a user data model data structure that represents a pattern of behavior of the user based on the monitored user state;
detect, by a user state change detection component executing within the health behavior change engine, a change in user state;
analyze, by a state change analysis component executing within the health behavior change engine, the change in user state to determine whether to activate a dialog;
generate, by a dialog generation component executing within the health behavior change engine, a health-based conversation dialog in response to the state change analysis component determining to activate the dialog; and
communicate, by a communication component executing within the health behavior change engine, with the user via the intelligent personal assistant using the health-based conversation dialog.
